SEQUENCE LISTING (3 sequences)

SEQ ID NO: 1 - DNA, 21 bp, Rattus norvegicus
  prim_transcript (1)..(21): antisense oligo for V-EGF
cggcaatagctgcgctggtag

SEQ ID NO: 2 - DNA, 16 bp, unknown
  unknown
tggacctggctttact

SEQ ID NO: 3 - DNA, 18 bp, Unknown
  unknown
tccctggttccccgaata

(19) United States
(12) Patent Application Publication
(10) Pub. No.: US 2008/0206341 A1
(43) Pub. Date: Aug. 28, 2008

(54) Lipid Nanoparticles as Vehicles for Nucleic Acids, Process for Their Preparation and Use

(76) Inventor: Maria Rosa Gasco, Torino (IT)

Correspondence Address: COLLARD & ROE, P.C., 1077 NORTHERN BOULEVARD, ROSLYN, NY, 11576, US

(21) Appl. No.: 11/629,141

(22) PCT Filed: Jun. 8, 2005
(86) PCT No.: PCT/EP2005/052647

§ 371 Date: Jul. 17, 2007

(30) Foreign Application Priority Data
Jun. 9, 2004 (IT) MI2004A001151

(51) Int. Cl. A61K 9/14; A61K 31/7052; C12N 5/06

(52) U.S. Cl. 424/489; 514/44; 435/375; 977/906

(57) ABSTRACT

The invention relates to solid lipid nanoparticles composed of lipid material and containing, as bioactive molecule, a nucleic acid, preferably an antisense oligonucleotide, preferably modified by chemical methods to achieve a greater resistance to endo- and exo-nucleases, and to the process for preparation of the nanoparticles. In the present invention, the efficiency of the delivery system represented by nanoparticles containing synthetic or natural polynucleotides allows the use of such system for transfection. The particles are especially effective in the treatment of diseases of the posterior segment of the eye (such as diabetic retinopathy, macular degeneration, etc.) and in angiogenesis.

### FIELD OF THE INVENTION

The technical field of the invention relates to the delivery of nucleic acids by means of nanoparticles having a lipid composition.

### PRIOR ART

Nucleic acid delivery technologies have been continuously developing and new methods for transfer to target cells are critical for the success of gene therapy. In fact, high efficiency and low toxicity of delivery systems are essential factors to make polynucleotide transfer feasible.

Until now, several chemical systems have been established that are based on lipids, cathionic vesicles, cathionic lipids, etc; however, the in vitro toxicity of such systems restricts their potential therapeutic use.

To date, viral and/or retroviral vectors are still the most efficient and least cytotoxic delivery systems, although certainly their use is not devoid of medium-term and long-term risks.

In the field of nucleic acid therapy, antisense therapy with oligonucleotides (AS-ODN), that are synthetic ribonucleic or deoxyribonucleic acid fragments which specifically bind their complementary messenger RNA thereby blocking translation of the corresponding protein, turned out to be a rather promising approach. However, once again, a wide use of these molecules in all possible fields of application is limited by their high susceptibility to degradation in biological fluids and in cellular systems, mainly due to the presence of exo- and endo-nucleases which hydrolyze phosphodiester bonds.

Additional limits to the use of these molecules relate to the problem of their poor diffusion through membranes, owing to the general strong ionic nature of nucleic acids, and to the fact that their cellular internalization depends on many variables, including temperature.

Therefore, different strategies have been proposed to reduce nucleic acid degradation, to increase their intracellular penetration and their release in the cytoplasm, for example by means of various carriers such as polymeric nanoparticles, liposomes etc.

In particular, it has been observed that ODN vehicled by nanoparticles made of polyalkylcyanoacrylate (Nanoparticulate systems for the delivery of antisense oligonucleotides, Advanced Drug Delivery Reviews (2001), 47 99-112 Lambert G, Fattal E., Couvreur P.) or poly(lactide-co-glycolide) (described in <<Nanoparticle formulation enhances the delivery and activity of a vascular endothelial growth factor antisense oligonucleotide in human retinal pigment epithelial cells>> J. Pharm. Pharmacol. (2003), 55, 1199-1206, Aukunuru I V, Ayalasomajula S P, Kompella U B) are protected from degradation and show the ability to penetrate different cell types.

Other liposome-based techniques have been described in several articles, including: Folia Morphol., (2003)62:397-9; <<Evaluation of transfection effectiveness using fluorescein labelled oligonucleotides and various liposomes>>, Surowiak P; and in: <<Associating oligonucleotides with positively charged liposomes; Cell Mol. Biol. Lett 2003; 8; 77-84; Jurkiewicz P., Okruszzek A., Hof M. Langner M; and in: <<A lipid based delivery system for antisense oligonucleotides derived from a hydrophobic complex>>, J. Drug Targeting 2002, 10; 615-23; Wong F M, Mac Adam S A, Kim A, Oja C, Ramsay E C, Bally M B.

Moreover, solid lipid particles have been described in EP 526666.

### SUMMARY OF THE INVENTION

The invention relates to nanoparticles having a diameter ranging from 80 to 400 nm, preferably ranging from 50 to 200 nm, consisting of lipid material and containing a nucleic acid as bioactive molecule. Said nucleic acid is preferably an antisense oligonucleotide that has been chemically modified in order to achieve greater resistance to endo- and exo-nucleases.

The efficiency of the delivery system represented by the nanoparticles of the invention, containing synthetic or natural polynucleotides, allows their use for transfection of target cells, preferably neoplastic or “normal” mammalian cells, even more preferably stem cells or cell lines.

Said particles proved to be especially effective in molecular therapy with antisense oligonucleotides (particularly for diseases of the posterior segment of the eye, such as diabetic retinopathy, macular degeneration, etc.), in angiogenesis, and in all those cases where the antisense approach already proved to be effective, at least in vitro.

Moreover, the formulation of nucleic acids incorporated in solid lipid nanoparticles (SLN) allows their administration through both systemic and topical routes.

### DETAILED DESCRIPTION OF THE INVENTION

The invention relates to solid lipid nanoparticles (SLN) containing nucleic acids, particularly polynucleotides and oligonucleotides, to the process for preparation of said-nucleic acid-containing nanoparticles and to the use of lipid particles to deliver polynucleotides or nucleic acids, preferably oligonucleotides and <<small interfering RNA>> (si RNA).

The preparation of solid lipid nanoparticles is carried out according to the following steps:

a microemulsion is prepared by heating until one or more lipids have been melted, optionally adding a surfactant, a solution comprising water, a nucleic acid and a co-surfactant, optionally a surface-active agent is prepared, and the two components are mixed at a temperature that is at least equal to the melting point of said lipid or lipids.

The so obtained hot-microemulsion has the following composition in weight:
lipid component, ranging from 5 to 42%, more preferably ranging from 10 to 20%,
water from 10 to 70%, more preferably from 25 to 65%,
surfactants from 8 to 35%, preferably from 12 to 20%,
co-surfactant from 5 to 30%,
nucleic acid (or nucleic acid solution) in an amount ranging from 0.1 to 6%,
optionally a nucleic acid counterion, as for example DC-cholesterol, cetylpyridinium chloride or bromide or a cationic lipid such as DOPE (dioleilphosphatidylethanolamine).

The hot-microemulsion is then dispersed in water at a temperature comprised from 2 to 8° C., with a dispersion ratio 1:1-1:10 (microemulsion:cold water), and is washed, for instance, by diafiltration with water. The water used for the washing step may comprise an amino acid, preferably a basic amino acid.

Alternatively, the hot-microemulsion can be added to a water-mixture equilibrated to a temperature equal to the temperature of the hot-microemulsion further comprising (in w.w): a co-surfactant (5-20%), a surfactant (3-15%), and optionally lipids (concentration 0-4%), and it is then dispersed in water at a temperature comprised between 2 and 8° C., as described above. Even in this case the dispersion can be washed, for instance by diafiltration with water. The water used for the washing step can contain an amino acid, preferably a basic amino acid, in a weight amount comprised between 0 and 2%.

Dried lipid nanoparticles can be obtained by a further step of freeze-drying, or desiccation by evaporation at low temperature or by spray-drying.

One or more substances suitable to sterically stabilize nanoparticles can be added to the hot-microemulsion, such as for instance: di-palmitoyl posphatidylethanolamine-PEG (PEG.750-2000),

diacyl-phosphadytilethanolamine pegylated with PEG (PEG M.W. 750-2000), stearate and fatty acids pegylated with polyethilene-glycol methylether (PEG M.W.750-2000).

The lipid components used in the process of the present invention are selected from the group consisting of:
triglycerides such as, for example, trilaurin, tricapryloin, tripalmitin, tristearin, diglycerides as, for example, dipalmitin and distearin, capric/caprilyc triglycerides (Mygliol®, Captex®, Labrafac®) monoglycerides such as glycerylmonostearate (Myvaplex®600) or glycerylpalmitostearate; particularly preferred are tripalmitin, glycerylmonostearate and palmitoylstearate
aliphatic alcohols, for instance cetyl alcohol, stearyl alcohol;
medium-long chain carboxylic fatty acids (C₁₀-C₂₂), and their esters with polyalcohols such as propylene glycol; particularly preferred are stearic acid (C18); palmitic (C16);
cholesterol and cholesterol esters such as cholesteryl hemisuccinate, cholesteryl butyrate, cholesteryl-palmitate.

The surface-active agents or surfactants are preferably selected from the group consisting of:
lecithins (e.g. Lipoid 75, Epikuron 200) or other types of phospholipids;
bile salts and bile acids, e.g. sodium glycocholate and glycocholic acid, sodium taurocholate and taurocholic acid, taurodeoxycholate, dioctylsulphosuccinate (AOP);
Tween®20, Tween®40, Tween®80,

Particularly preferred are lecithins and phospholipids.

Co-surfactants are selected from the group consisting of: low molecular weight alcohols and glycols as, for example, propanol, isopropanol, butanol, hexanol, short chain fatty acids, such as, for example, octanoic acid or butyric acid, phosphoric acid monoesters, benzyl alcohol and bile salts such as taurocholate. Short chain aliphatic acids and bile salts are particularly preferred.

Particularly preferred counterions include cetylpyridinium chloride, DC-cholesterol or cationic lipids, such as DOPE.

Nucleic acids preferably have molecular weight lower than 50000 Daltons or even more preferably lower than 30000 Daltons, can be single or double stranded, can be deoxyribonucleotides or ribonucleotides. Preferably, nucleic acids are chemically synthesized oligonucleotides (ODN) that can be modified, for example labeled, preferably with fluorescein. Even more preferably, they are synthetized by means of phosphorothioate nucleotides.

Nucleic acids are preferably anti-sense oligonucleotides-that can specifically base pair to complementary mRNA and prevent mRNA translation and production of the corresponding protein.

According to a preferred embodiment, nucleic acids are small interfering RNAs (si RNA) having a mechanism of action as described, for instance, in Sioud M. Trends in Pharmacological Sciences, 2004 25:22-28.

The water used for microemulsion is injectable water.

Lipid nanoparticies prepared according to the invention have the following characteristics:
penetrate the blood-retinal barrier, thus, when administered through the topical route, can reach the posterior segment of the eye and deliver nucleic acids. This opens a therapeutic prospect for therapy of diseases of the posterior segment of the eye, such as for example macular degeneration or diabetic retinopathy, as well as tumor pathologies;
protect the integrity of the incorporated nucleic acid from the action of degrading enzymes (e.g. nucleases), that are present in biological fluids, and can be administered through the parenteral route, preferably by intravenous injection;
are able to deliver nucleic acids to eukaryotic cells, preferably mammalian cells, both in vitro and in vivo;
penetrate the blood-brain barrier, thereby delivering nucleic acids directly to the brain microvasculature.

The nanoparticles of the invention, containing nucleic acids (also called polynucleotides or oligonucleotides in the present invention), are claimed for use in the treatment of cerebral and ophthalmic diseases, including tumor pathologies, and particular in diabetic retinopathy and in macular degeneration.

The nanoparticles of the invention are suitable for preparation of compositions for topical or parenteral use. For parenteral use, said nanoparticles are administered in doses corresponding to an amount of oligonucleotide (ODN) ranging from 0.01 to 5 mg/kg of body weight, more preferably ranging from 2 to 3 mg/kg.

In the compositions for topical ocular administration, the concentration of nanoparticles in the isotonic aqueous dispersion ranges from 1 to 25% weight/volume. Moreover the nanoparticles of the invention optionally contain an amount of viscosizing substance ranging from 0.1 to 0.4%.

In a preferred embodiment, said compositions, including antisense oligonucleotides, are used for the treatment of diseases associated with expression or overexpression of a gene coding one or more proteins.

According to a further aspect, the invention relates to the use of solid lipid nanoparticles for incorporation and delivery of nucleic acids. Such delivery is directed to target cells comprising: eukaryotic cells, such as mammalian cells, cell lines, stem cells, primary cell lines, and can lead to transfection or cell transformation in vitro or ex-vivo. Therefore, according to this aspect, the invention relates to a kit for transfection of eukaryotic cells, comprising the solid lipid nanoparticles of the invention and suitable diluents and/or cell washing buffers.

Furthermore, owing to their carrier properties and to their ability to protect incorporated nucleic acids said nanoparticles are suitable for preparation of a medicament for delivery of nucleic acids in vivo.

Therefore, according to this aspect, the invention relates to a method for gene therapy in subjects affected by diseases, e.g. tumor pathologies, preferably of the central nervous system, comprising parenteral administration of said nanoparticles in an amount corresponding to 0.01-5 mg of oligonucleotide (ODN) per kilogram of body weight, or more preferably ranging from 2 to 3 mg/kg. Said administration is preferably by the intravenous route.

Moreover, the invention includes a therapeutic method for treatment of ophthalmic diseases, by topical ocular administration of an amount of solid lipid, nanoparticles corresponding to an amount of oligonucleotide comprised between 0.01 and 5.0 mg for each eye.

Experimental Part

### EXAMPLE 1

### Preparation of Nanoparticles of Different Composition Containing Phosphorothioate Oligonucleotides

Stearic-acid: (39%) has been melted at 70° C., while mixing with Epikuron 200 (24%). An aqueous solution (24%), containing 10% sodium taurocholate and 3% phosphorothioate antisense oligonucleotide with sequence CGGCAATAGCTGCGCTGGTAg (M.W. 6519) has been added. A clear hot-system was obtained, which constituted mixture 1. The so obtained mixture (that is clear at hot temperature) was added slowly to mixture II composed of Epikuron 200 (6%), taurocholate (13%), isopropilic alcohol (3%), water (78%) (mixture II), always at the same temperature (70°). All percentages shown were in w/w.

The mixing ratio between mixture I and mixture II was 1:4.2-4.4. The clear system has been then dispersed in water in a 1:5 ratio at 2-3° C.

The dispersion has been washed three times by diafiltration. By this means, lipid nanoparticles containing ODN have been obtained, with an average diameter of 75 nm and an oligonucleotide titer in the dispersion of 0.55 mg/ml.

### EXAMPLE 2

### Preparation of Nanoparticles of Different Composition, Containing Phosphorothioate Oligonucleotides

Stearic acid (31.9%) and Epikuron 200 (22.5%) have been melted, and octanoic acid (6.4%) has been added. A mixture of isopropilic alcohol (14%), as ODN (3.1%) solubilized in water (20.6%) and sodium glycocholate (1.5%) has been added at hot temperature. A clear hot-system was obtained (mixture I) that has been added slowly, at 70° C., to mixture II, composed as follows: Epikuron 200 (5.8%), sodium glycocholate (12.8%), isopropilic alcohol (6%), water (75.4%). All percentages were in w/w. The mixing ratio between mixture I and mixture II was 1:4.1-1:4.3. A clear system was obtained that has been dispersed in water in a 1:9 ratio at a temperature of 2-3° C., under stirring. Dispersed lipid nanoparticles were obtained (average diameter 142 nm). The dispersion has been washed three times by diafiltration.

After washing, a dispersion was obtained, containing an oligonucleotide concentration of 0.6 mg/mL

### EXAMPLE 3

### Preparation of Nanoparticles of Different Composition, Containing Phosphorothioate Oligonucleotides

Stearic acid (32.2%) and Epikuron 200 (22.4%) have been melted; octanoic acid (6.4%) has been added to the melted mixture, followed by addition, always at hot temperature, of isepropilic alcohol (16.0%), sodium taurocholate (1.6%), and antisense oligonucleotide AS-ODN with the following sequence: cGGCAATAGCTGCGCTGGTAg (M.W. 6519) (2.2%) solubilized in water (19.2%), thus obtaining a clear hot-mixture (mixture I).

Mixture I has been slowly added, at hot temperature, to mixture II composed of Epikuron 200 (5.8%), sodium glycocholate (13.2%), isopropilic alcohol (4.5%) and water (76.5%), thus obtaining a clear hot-mixture (mixture II). This mixture was then dispersed in cold water (2-3° C.) in a ratio 1:9, under stirring: a lipid nanoparticle dispersion was obtained (average diameter: 110 nm). The dispersion has been washed three times: the oligonucleotide titer turned out to be 0.83 mg ODN/mL.

### EXAMPLE 4

### Use of Nanoparticles Containing Anti-VEGF Oligonucleotides in Mammalian Cells

The solid lipid nanoparticles prepared according to the previous example have been tested on rat C6 glioma cells.

For the purpose of this experiment, a 100 nM (antisense AS-ODN) dispersion of nanoparticles carrying the oligonucleotide, and a 100 μM solution of the same antisense oligonucleotide in non-carriered form have been prepared; treatments were made on cells under both standard (5-10% CO₂ atmosphere) and hypoxic conditions.

The analysis was performed by comparison with the results obtained from C6 glioma cells that were not treated with the antisense.

The two formulations of antisense oligonucleotides,—i.e. 100 M As-ODN solution and 100 nM As-ODN-SLN dispersion—have been incubated with cells for 24, 36, 48 hours.

VEGF mRNA expression has been analyzed by both RT-PCR and Western blot (semiquantitative) performed on both homogenates and supernatants (protein isoforms have been also analyzed).

Both types of analysis shown that VEGF expression was markedly reduced following treatment with anti-VEGF antisense oligonucleotide incorporated into nanospheres. From a quantitative point of view, VEGF expression was completely blocked by SLN at 100 nM concentration, while VEGF expression was still present following incubation of cells with the aqueous solution containing a 1000-fold higher Antisense concentration.

### EXAMPLE 5

### Preparation of Nanoparticles Containing Phosphorothioate Oligonucleotides, Obtained in Presence of DC-Cholesterol

Cholesterylpalimitate (7.3%) has been melted together with DC-cholesterol (3β-(N-(N′,N′-dimethylaminoethane)carbamoyl), cholesterol hydrochloride 0.8%, and EPIKURON (5.5%); a solution at the same temperature as the melting temperature, composed of anti-VEGF (0.1%) in water (73.0%) and sodium taurocholate (13.3%), has been added to the mixture: A clear hot-system has been obtained, that was dispersed in a 1:4 ratio in water, at 2-3°. A dispersion of lipid nanoparticles was obtained, and said nanoparticles were washed three times by diafiltration, thus obtaining a dispersion having a As-ODN titer of 0.15 mg/ml.

### EXAMPLE 6

### Preparation of Nanoparticles Containing Modified or Derivatized Oligonucleotides

In an early phase, stearic acid (27.3%) was melted at hot temperature (70° C.); Epikuron 200 (34.2%) was added. Butyric acid (23.4%), butanol (4.9%) and an aqueous solution containing 4% phosphorothioate AS-ODNA with sequence cGGCAATAGCTGCGCTGGTAg (M.W. 6519) (10.2%) were then added.

A clear hot-system was obtained, which constituted mixture I. Such mixture (that is clear at hot temperature) has been added slowly, always at the same temperature (70° C.), to a mixture composed of Epikuron 200 (4.1%), Taurocholate (4.1%), butyric acid (9.8%), water (82.0%) (mixture II). All percentages shown were in w/w. The mixing ratio between mixture I and mixture II was 1:8.2-1:8.4.

The mixture was slowly added until a clear system was obtained at a temperature of about 70° C.; the clear system was then dispersed in a 1:4 ratio in water at 2-3° C. The dispersion has been washed three times by diafiltration. By this means, lipid nanoparticles were obtained that contained 0.10 mg/ml AS-ODN in the dispersion. Nanoparticles were then washed with aqueous solution containing 0.2% lysine.

### EXAMPLE 7

### Preparation of Nanoparticles Containing Modified Oligonucleotides Obtained in the Presence of Cetylpyridinium

Solid lipid nanoparticles were prepared that contained oligonucleotides modified by fluorescein coupling.

In particular, the phosphorothioate oligonucleotide used had the following sequence: 5′-Fluorescein-Tgg-Ac-CTg-gCT-TTA-CTg as detailed below: stearic acid (8.0%) has been melted and Epikuron 200 (4.3%) has been added, then sodium taurocholate (14.6%) and an aqueous solution (72.4%), containing 0.18% As ODN, have been added to the mixture; 0.7% cetylpyridinium chloride has been added to the so obtained clear hot-system (about 70° C.).

After stirring, the clear system has been dispersed in water at 2-3° C. in a 1:4 ratio. Washing was then performed by diafiltration, thus obtaining a lipid nanoparticle dispersion containing 0.02 mg ODN per ml of dispersion.

In a subsequent test, a different phosphorothioate oligonucleotide sequence was used: 5′-TCC-CTg-gTT-CCC-CgA-ATA, prepared as follows: stearic acid (8.1%) has been melted and Epikuron 200 (4.3%), sodium taurocholate (14.6%), water (72.8%), containing 0.20% As-ODN and cetylpyridinium chloride (0.2%), have been added. A clear hot-system was obtained that, upon dispersion in water at a 1:3 ratio, yielded lipid nanoparticles. Such lipid nanoparticles have been then washed by diafiltration, obtaining a titer of 0.025 mg of As-ODN per ml of dispersion.

1-46. (canceled)

47. Solid lipid nanoparticles comprising:
one or more lipids selected from the group comprising: trilaurin, tricapryloin, tristearin, tripalmitin, capriclcapylic triglycerides, dipalmitin, distearin, glyceryl monostearate, glyceryl palmitostearate, cetyl alcohol, stearilic alcohol, fatty acids having a C₁₀-C₂₂ chain, cholesteryl hemisuccinate, cholesteryl butyrate and cholesteryl palmitate
a surfactant,
a co-surfactant,
a nucleic acid,
water,
optionally nucleic acid counterions and/or lipid particle stabilizers
wherein the nucleic acid is incorporated in the solid lipid nanoparticle.

48. Nanoparticles according to claim 47, having an average diameter comprised from 50 to 400 nm.

49. Nanoparticles according to claim 48, wherein their average diameter is comprised from 80 to 200 nm.

50. Nanoparticles according to claim 47, wherein said nucleic acids are oligonucleotides.

51. Nanoparticles according to claim 47, wherein said nucleic acids are si RNAS.

52. Nanoparticles according to claim 50, wherein the length of said oligonucleotides ranges from 10 to 50 bases.

53. Nanoparticles according to claim 50, wherein said oligonucleotides are antisense oligonucleotides.

54. Nanoparticles according to claim 53, wherein said antisense oligonucleotide is an anti-VEGF oligonucleotide.

55. Nanoparticle according to claim 54, wherein said anti-VEGF oligonucleotide has the following sequence: SEQ ID NO. 1.

56. Nanoparticle according to claim 50, wherein nucleic acid counterions are selected between cetylpyridinium and DC-cholesterol.

57. Nanoparticle according to claim 51, wherein nucleic acid counterions are selected between cetylpyridinium and DC-cholesterol.

58. Nanoparticles according to claim 47, wherein said stabilizers are selected from the group comprising: di-palmitoyl phosphatidyl ethanolamine-PEG (PEG M.W. 750-2000), diacylphosphadytilethanolamine pegylated with PEG (PEG M.W. 750-2000), stearate and fatty acids pegylated with polyethilene-glycol methylether (PEG M.W.750-2000).

59. Process for preparation of solid lipid nanoparticles containing a nucleic acid, comprising the following steps:
a) preparation of a hot-microemulsion by mixing, at hot temperature, a mixture composed of one or more melted lipids, optionally containing one or more co-surfactants, with an aqueous mixture containing a surfactant, a nucleic acid and optionally one or more co-surfactants, at a temperature equal to or higher than the melting temperature of said lipids,
b) dispersion of the-microemulsion prepared at hot temperature in a), in water at a temperature ranging from 2 to 8° C. with a 1:1-1:10 dispersion ratio (microemulsion:cold water) and washing the dispersion with water optionally containing an amino acid,
c) optional obtainment of dried lipid nanospheres by freeze-drying, desiccation by evaporation at low temperatures or by spray-drying.

60. Process for preparation of solid lipid nanoparticles containing nucleic acid, comprising the following steps:
a) preparation of a microemulsion by mixing, at hot temperature, a mixture composed of one or more melted lipids and optionally containing one or more co-surfactants, with an aqueous mixture containing a nucleic acid, one or more surfactants, and optionally one or more co-surfactants, at a temperature that is equal to or higher than the melting temperature of said lipids; a′) mixing said hot-microemulsion, at hot temperature, with a mixture composed of water, a surfactant, a co-sur[actant and optionally a lipid, in a ratio from 1:1 to 1:10, at a temperature at least equal to the melting temperature of said lipids, in order to obtain a clear hot-microemulsion;
b) dispersing the clear hot-microemulsion prepared in a′) in water at temperature ranging from 2 to 8° C. using a dispersion ratio 1:1-1:10 (microemulsion:cold water) and washing the dispersion with water optionally containing an amino acid,
c) optionally obtaining dried lipid nanoparticles by freeze-dying, desiccation by evaporation at low temperatures or by spray-drying.

61. Process according to claim 59, wherein a counterion is alternately added to steps a, a′, b, or c) of said process.

62. Process according to claim 60, wherein a counterion is alternately added to steps a, a′, b, or c) of said process.

63. Process according to claim 59, wherein said microemulsion has the following composition in weight:
one or more lipids in an amount from 5 to 42%, more preferably from 10 to 20%,
water from 10 to 70%, preferably 25-65%,
surfactant 8-35%, preferably 12-20%, co-surfactant 5-30%,
a nucleic acid in amount from 0.1 to 6%.

64. Process according to claim 60, wherein said microemulsion has the following composition in weight:
one or more lipids in an amount from 5 to 42%, more preferably from 10 to 20%,
water from 10 to 70%, preferably 25-65%,
surfactant 8-35%, preferably 12-20%,
co-surfactant 5-30%,
a nucleic acid in amount from 0.1 to 6%.

65. Process according to claim 59, wherein said lipids are selected from the group consisting of: tri!aurin, tricapryloin, tristearin, tripalmitin, capric/caprylic triglycerides, dipalmitin, distearin, glyceryl monostearate, glyceryl palmitostearate, cetyl alcohol, stearilic alcohol, fatly acids having a C₁₀-C₂₂ chain, cholesteryl hemisuccinate, cholesteryl butyrate and cholesteryl palmitate.

66. Process according to claim 60, wherein said lipids are selected from the group consisting of: trilaurin, tricapryloin, tristearin, tripalmitin, capric/caprylic triglycerides, dipalmitin, distearin, glyceryl monostearate, glyceryl palmitostearate, cetyl alcohol, stearilic alcohol, fatty acids having a C₁₀-C₂₂ chain, cholesteryl hemisuccinate, cholesteryl butyrate and cholesteryl palmitate.

67. Process according to claim 61, wherein said counterion is selected from the group consisting of DC-cholesterol, cetyl pyridinium chloride or bromide or a cationic lipid such as DOPE (dioleilphosphatidylethanolamino).

68. Process according to claim 62, wherein said counterion is selected from the group consisting of DC-cholesterol, cetyl pyridinium chloride or bromide or a cationic lipid such as DOPE (dioleilphosphatidylethanolamine).

69. Nanoparticles obtainable by the process according to claim 59.

70. Nanoparticles obtainable by the process according to claim 60.

71. Pharmaceutical composition comprising, as the active ingredient, lipid nanoparticles containing nucleic acids according to claim 47, in an isotonic aqueous dispersion.

72. The composition according to claim 71, wherein said nucleic acids are selected between antisense oligonucleotides and siRNA.

73. The composition according to claim 71, for intravenous or topical administration.

74. The composition according to claim 73, for topical use in the treatment of ophthalmic or brain diseases.

75. The composition according to claim 74, wherein said ophthalmic diseases are diabetic retinopathy and macular degeneration.

76. The composition according to claim 74, wherein said diseases are associated with expression or overexpression of a gene coding one or more proteins.

77. The composition according to claim 74, wherein said aqueous dispersion contains in addition a viscosizing substance.

78. The composition according to claim 73 for intravenous administration.

79. A method for incorporating nucleic acid into solid lipid nanoparticles wherein the process according to claim 59 is used.

80. The method according to claim 79 for nucleic acid delivery.

81. The method according to claim 80, wherein said delivery is carried out in vitro or ex-vivo.

82. The method according to claim 81, wherein said delivery is carried out in a target cell wherein said target cell is selected from the group consisting of: a eukaryotic cell such as a mammalian cell, a cell line, a stem cell, a primary cell.

83. The method according to claim 82 for cell transfection or cell transformation in vitro.

84. Kit for eukaryotio cell transfection comprising the solid lipid nanoparticles, according to claim 47, and suitable diluents and/or physiological washing buffers.

85. Kit for eukaryotic cell transfection comprising the solid lipid nanoparticles according to claim 69, and suitable dilutents and/or physiological washing buffers.

86. Method for gene therapy in subjects in need of gene-therapy wherein gene delivery is effected by the nanoparticles according to claim 47 and wherein said nanoparticles are administered by the parenteral route in an amount corresponding to 0.01 and 5 mg of oligonucleotide per kilogram of body weight.

87. Method according to claim 86, wherein said administration is by the intravenous route.

88. Method according to claim 86, wherein said disease is a tumor.

89. A therapeutic method for the treatment of ophthalmic and/or brain diseases, in subjects affected by at least one of such diseases, consisting in administration, through the topical ocular route, of a pharmaceutical composition according to claim 69 comprising effective amounts of solid lipid nanoparticles.

90. Therapeutic method according to claim 89, comprising the administration of an amount corresponding to 0.01-5.0 mg of oligonucleotide for each eye.

91. Therapeutic method according to claim 89, wherein said diseases are: macular degeneration, diabetic retinopathy, tumor pathologies of the central nervous system.
